(19) United States
(12) Patent Application Publication
(10) Pub. No.: US 2009/0149830 A1
(43) Pub. Date: Jun. 11, 2009

(54) Intelligent needle technology for acupuncture and injection of cosmetic preparations subcutaneously

(76) Inventor: Donald Spector, New York, NY (US)

Correspondence Address: COLLARD & ROE, P.C., 1077 NORTHERN BOULEVARD, ROSLYN, NY, 11576, US

(21) Appl. No.: 12/313,706

(22) Filed: Nov. 24, 2008

Related U.S. Application Data
(60) Provisional application No. 61/012,137, filed on Dec. 7, 2007.
(60) Provisional application No. 61/012,174, filed on Dec. 7, 2007.

(51) Int. Cl. A61M 5/32; A61M 5/20; A61M 5/44

(52) U.S. Cl. 604/506; 604/195; 604/151; 604/113

(57) ABSTRACT

Intelligent needles comprise a needle for insertion into the skin to deposit a substance that causes plumping of the skin. The penetration depth of the needle may be adjusted automatically in conjunction with a sensor mechanism that determines a depth the needle can penetrate without contacting blood vessels. Methods of making and using the same for acupuncture and subcutaneous injection are also provided.

### STATEMENT OF RELATED CASES

This application claims the benefit of priority under 35 U.S.C. § 119(e) to U.S. Provisional Patent Application Ser. No. 61/012,137, filed Dec. 7, 2007, and U.S. Provisional Patent Application Ser. No. 61/012,174, filed Dec. 7, 2007, both of which are hereby incorporated by reference in their entireties.

### FIELD OF THE INVENTION

This application generally relates to the field of acupuncture. More specifically, this application relates to acupuncture and intelligent needles for inserting substances subcutaneously for cosmetic purposes.

### BACKGROUND

Acupuncture has been used in facial rejuvenation to stimulate circulation, thereby reducing lines and wrinkles of aging or that are the result of injury. However, acupuncture does not currently include application of substances that may cause the skin to “plump” or swell because these substances should be placed below the skin, but not in a blood vessel. The swelling produced by the implanted substance can result in the reduced appearance of wrinkles.

Therefore, there exists a need for apparatus and methods to administer substances by acupuncture that are effective to cause swelling in the region of application.

### SUMMARY

This patent application relates to advances that have been made in the use of sensors and very thin needles that are presently employed to draw blood, many in the field of diagnostics.

In the development of blood capturing technologies, which are very important for diabetics, there is often a problem of numbing, associated with too many tests in a particular area. In order to solve this problem, several companies have developed thin needle technology and also have used measurement sensors to determine the minimal amount of depth needed in order to obtain a blood sample. The purpose of this equipment is to leave no traces on the skin, reduce pain and prevent associative physiological problems during diagnostic procedures. Suitable sensors of this type are known to those skilled in the art. The prior art thin sensor needles rely on a vacuum draw to capture blood.

Various embodiments of the invention invert this process and, instead of having the vacuum draw associated with the sensor needles, have a pumping mechanism associated with the sensor and the needles. The purpose of the sensors, in this case, is the opposite of that in the diagnostic procedures. By aspects of the present invention, blood vessels can be avoided and the needle can stay within the confines of the skin layers, without going into any capillaries. Therefore, the sensors would set or reset the needles to go to a thinner level than would be used in the diagnostic equipment.

Other aspects of the invention are directed to methods and apparatus to apply materials, such as silicone, under the skin, without entering the blood stream. This can be used to puff up the skin to avoid wrinkles for cosmetic purposes. Even though these materials will be absorbed or released from the skin in several days, this process will provide immediate cosmetic benefit at a lower cost than Botox or other injections that are presently used for cosmetic purposes.

Additional aspects of the invention are directed to techniques for using acupuncture to reduce lines and wrinkles of aging or that are the result of injury. While acupuncture has been used in facial rejuvenation to stimulate circulation, the presence of a compound to inflate the skin, such as silicone or a stimulant, is not currently part of the practice of Acupuncture as it is used.

Nevertheless, those trained in the techniques of needling and acupuncture are trained to place needles under the skin painlessly and without bleeding. By providing either coatings on the needles of substances that react when they are placed under the skin or deposits of such substances under the skin either through the manipulation of the needle, temperature or other mechanical means, the acupuncturist can deliver product exactly where it is needed for a person who wants to reduce the effects of aging or other cosmetic reasons.

This technique involves substances that are non-toxic and can remain in place. The expertise of the acupuncturist in directing these materials subcutaneously can result in immediate results. The procedure is both simple and inexpensive.

Various aspects of the invention are directed to the addition of substances, including but not limited to silicone, that have the effect of “plumping” the skin. These substances can be either coated on the acupuncture needles or made a part of the needle (i.e., a filled syringe needle) such that they can be released by the acupuncturist either through mechanical manipulation of the needle, interaction with the body, heat applied from another source or a mechanical system operating with or contained within the needle. While this represents several embodiments of the concept, it is understood that there are many other ways in which these materials can be released subcutaneously, without departing from the spirit of the invention. Additional aspects are directed to processes of using acupuncture as a delivery system for these substances which cosmetically reduce wrinkles in the skin.

Embodiments of the invention are directed to an intelligent needle for subcutaneous injection. The intelligent needle comprises a housing and a movable needle within the housing. The needle has a tip and is movable between a retracted position, where the needle tip is within the housing, to an extended position, where the needle extends from within the housing. The intelligent needle also has an adjustment mechanism coupled to the needle. The adjustment mechanism is operative to move the needle between the retracted position and the extended position. The intelligent needle also has a sensor operatively coupled to the adjustment mechanism. The sensor is adapted to determine a penetration depth, which is an amount the needle can penetrate the skin while avoiding blood vessels.

The intelligent needle can further comprise a pump mechanism coupled to the needle and sensor. The pump mechanism is operable to pump contents of the needle into the skin upon receiving a signal from the sensor.

The intelligent needle can further comprise a substance that causes plumping of the skin. In one embodiment, the substance is silicone. In one embodiment, the substance coats the needle. In other embodiments, the needle is prefilled with the substance.

The substance may be released from the needle upon one or more of physical manipulation of the needle, mechanical manipulation of the needle, interaction with the body and applied heat.

Other embodiments of the invention are directed to methods of administering acupuncture therapy. The methods comprise inserting a needle into the skin of a patient; and depositing a substance under the skin of the patient, the substance operative to cause plumping of the skin.

In detailed aspects, the needle is inserted into the skin to a depth less than that which would be required to contact a blood vessel. In further detailed aspects, the insertion depth is determined by a sensor. In other embodiments, the substance is automatically deposited by a pump coupled to the needle, the pump being activated by the sensor detecting the needle inserted to the desired depth.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows an intelligent needle in accordance with one or more embodiments of the invention;

FIG. 2 shows an intelligent needle in accordance with one or more aspects of the invention;

FIG. 3 shows an intelligent needle in accordance with one or more aspects of the invention;

FIG. 4 shows an intelligent needle with a coated needle tip in accordance with aspects of the invention; and

FIG. 5 is an expanded view of the coated needle tip of FIG. 4.

### DETAILED DESCRIPTION

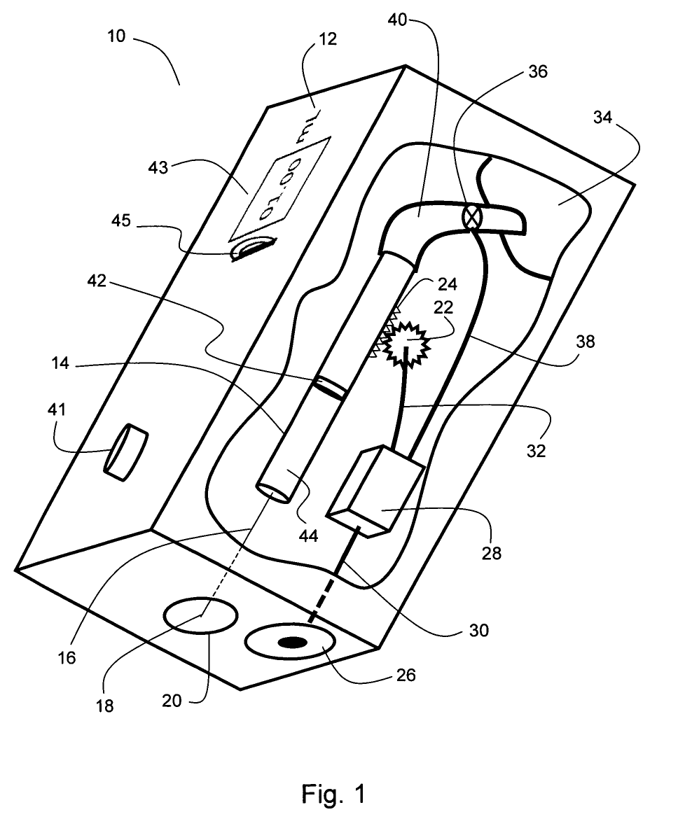

One or more embodiments of the invention are directed to intelligent needles for subcutaneous injection. The intelligent needle 10 of FIG. 1 has a housing 12 which can be held by a person. A syringe 14, or similar mechanism, is located within the housing 12 and has a needle 16 attached thereto. The needle 16 can be replaceable using, for example, Luer® type connectors, or other mechanisms. The needle can also be fixed in various embodiments. The needle 16 is of suitable gauge and sharpness to allow for subcutaneous insertion. The needle tip 18 can extend from within the housing 12 through a hole 20 in the housing 12.

The degree to which the needle tip 18 extends from the housing 12 can be controlled by an adjustment mechanism 22, 24. The adjustment mechanism shown in FIG. 1 consists of a rack 24 and pinion 22 type design. Other suitable mechanisms include, but are not limited to, screw threads, stepper motors, servos, solenoids, and piezoelectric devices. The adjustment mechanism 22, 24 is operative to move the syringe 14, and therefore the needle 16 and needle tip 18, between a retracted position and extended positions. In the retracted position, the needle tip 18 is within the housing 12 or flush with the housing 12. In the extended positions, the needle tip 18 extends from the housing by an amount determined by the adjustment mechanism 22, 24 and associated sensor 26.

A sensor 26 is coupled through a processor 28 to the adjustment mechanism 22, 24. The sensor 26 is connected to the processor 28 by connection 30. The processor 28 is connected to the adjustment mechanism 22, 24 by adjustment connection 32. The sensor 26 is adapted to determine the depth that the needle tip 18 can be inserted into the skin without contacting a blood vessel. The sensor 26 measures the skin properties and transmits this information to the processor 28 through connection 30. The processor 28 calculates the proper depth and causes the adjustment mechanism 22, 24 to move the syringe 14 so that the needle tip 18 extends from the housing 12 by the calculated amount.

According to some aspects of the invention, the intelligent syringe includes a pump mechanism. This mechanism can be a mechanical pump, air bladder, or other suitable mechanism operable to pump the contents of the syringe into the skin. FIG. 1 shows an air bladder 34 type mechanism, which may be useful in single use devices. The air bladder 34 may also be refillable. A valve 36 can receive a signal from the processor 28 over connection 38 indicating that the needle tip 18 is in position. The sensor 26 may be adapted to sense when the housing 12 and needle tip 18 contact the skin and transmit a signal to the processor 28. Upon receiving a signal from the processor 28, the valve 36 opens, thereby releasing pressure from the air bladder 34. The air travels through tubing 40 into the syringe 14 causing the plunger 42 to travel toward the needle tip 18, expelling the substance 44 within the syringe. The valve 36 may be opened for a short duration so that only a small amount of the substance 44 is expelled. This intelligent needle 10 can therefore be used for repeated injections.

Alternatively, the housing 12 of some aspects includes an actuator 41, which can be a simple button. Pressing the actuator 41 sends a signal to the processor 28 through a connection (not shown). A signal can then be sent from the processor 28 to activate the valve 36.

Some aspects of the invention, as shown in FIG. 1, include a mechanism adapted to allow the user to adjust the volume of the substance 44 delivered. The mechanism may include a display 43 and an adjustment wheel 45. Turning the adjustment wheel 45 can set the volume to be delivered, with the delivery volume being shown on the display 43. The display can be, for example, a simple set of numerical wheels, as is common on laboratory micro-pipettes, or a liquid crystal display. The delivery volume is transmitted to the processor 28 which can then transmit one or more signals to the valve 36 instructing the valve 36 to open and close.

In some aspects, the substance 44 is pre-filled in the syringe 14. Other embodiments have the substance 44 separate from the syringe 14 so that the syringe 14 can be filled with the substance 44 or the needle tip 18 can be dipped into the substance 44.

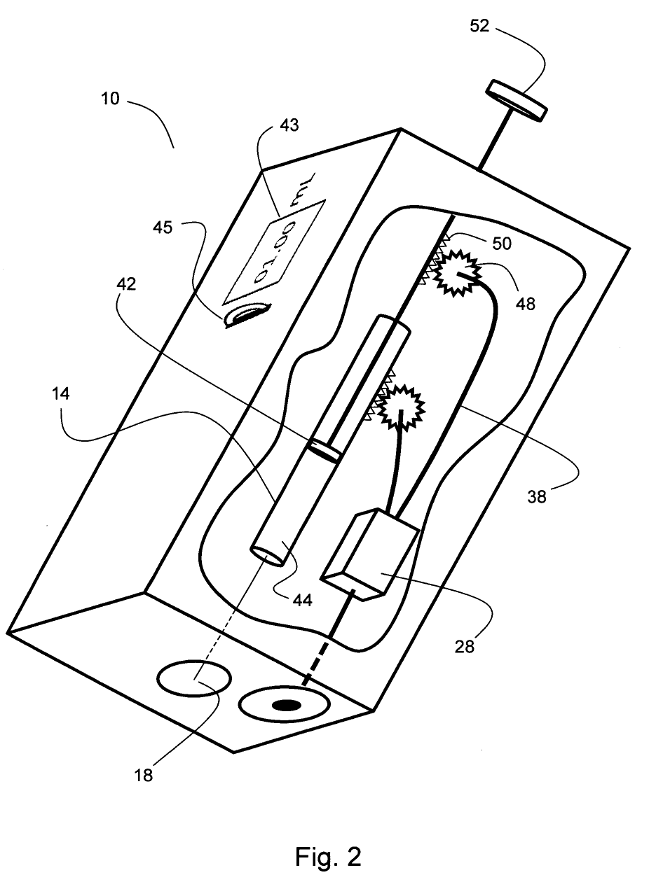

FIG. 2 shows another embodiment of the intelligent needle 10, where the contents of the syringe 14 can be ejected manually or automatically. Once the needle tip 18 has been inserted to the desired depth, the processor 28 sends a signal through connection 38 to an injection mechanism 48, 50. The injection mechanism 48, 50 is illustrated as a rack 50 and pinion 48 type mechanism but can be any suitable system. Non-limiting examples include stepper motors, screw threads, servos, piezoelectric devices an solenoids. The injection mechanism 48, 50 is operative to cause the plunger 42 in the syringe 14 to be pushed toward the needle tip 18, thereby emptying the substance 44 into the patient. Again, the plunger 42 may only be moved a small amount each time the injection mechanism 48, 50 is activated, allowing the intelligent needle 10 to be used multiple times on a single patient.

Alternatively, the intelligent needle 10 may include an externally accessible plunger rod 52. This allows the user to depress the plunger rod 50, forcing the plunger 42 toward the needle tip 18 and expelling the substance 44. The manual plunger rod 52 and injection mechanism 48, 50 can be included and operate independently or cooperatively. For example, the injection mechanism 48, 50 may be triggered by pressure on the plunger rod 52.

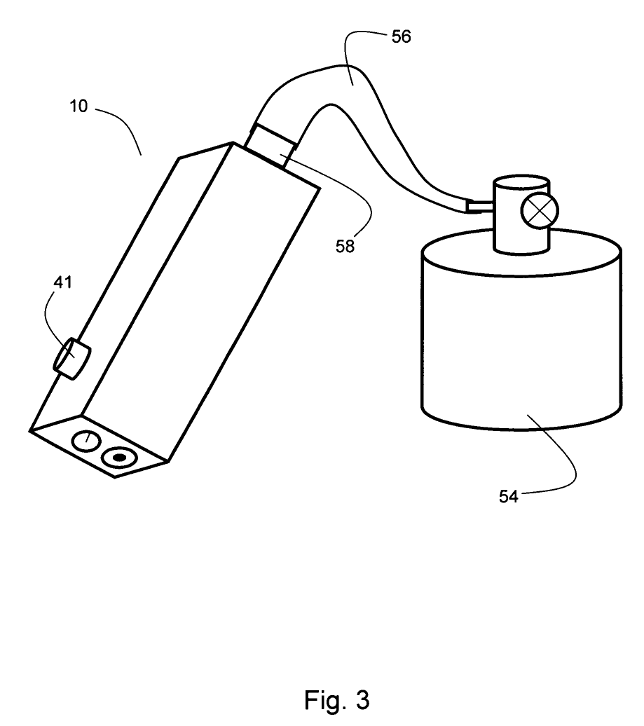

FIG. 3 shows an aspect of the invention where the intelligent needle 10 is attached to an external pressure source 54, such as a standard gas cylinder or compressor. A tube 56 can be connected to an inlet valve 58 on the intelligent needle 10. Upon receiving a signal from the processor (not shown), the inlet valve 58 can open for a specified period of time, allowing pressure to push the substance from the syringe. The inlet valve 58 may also be manually triggered by an actuator button 41 on the syringe. The length of time that the inlet valve 58 is open can be customized depending on the desired application.

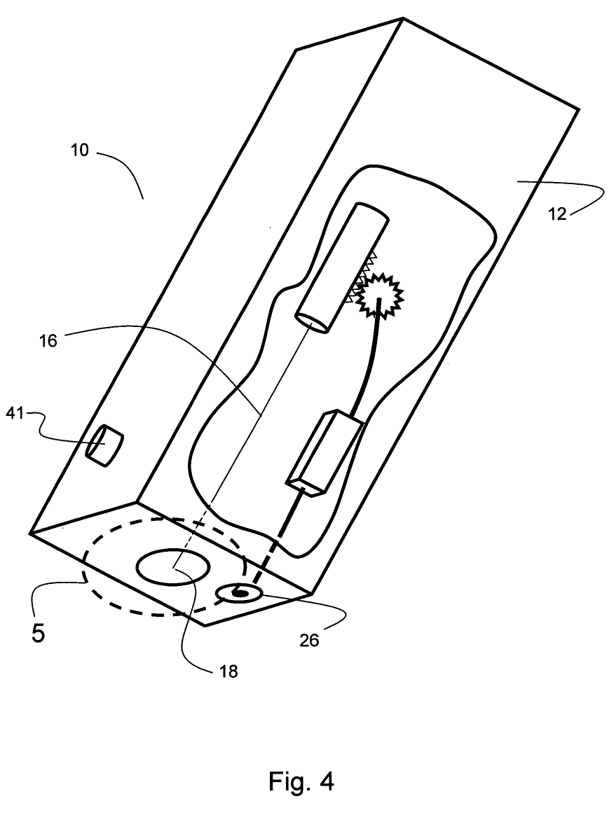

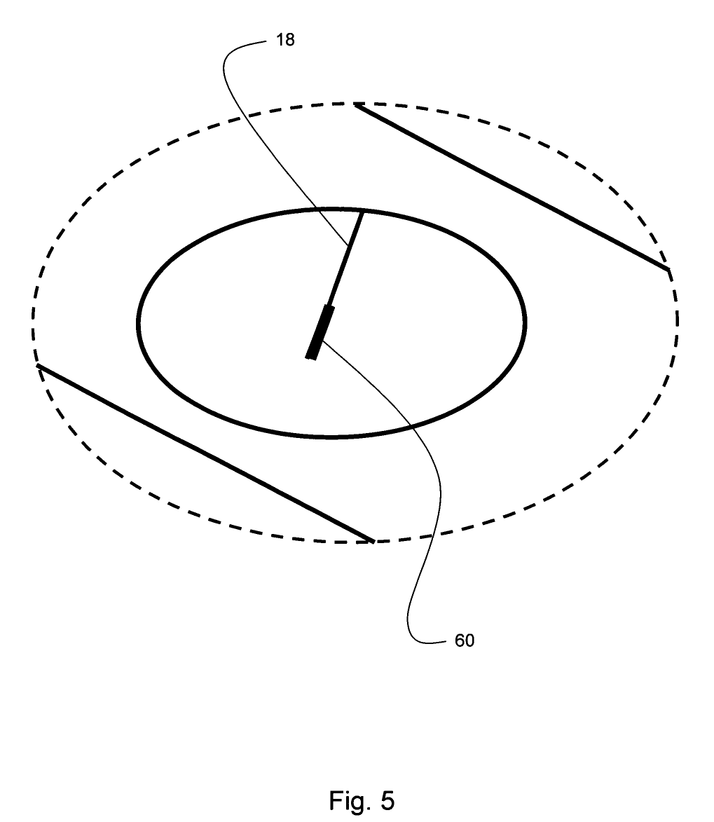

FIG. 4 shows another embodiment of an intelligent needle 10 where a syringe is omitted and replaced with a needle 16. The needle 16 has a tip 18 which can be adjusted to extend from or be recessed within the housing 12 using the previously described or similar mechanisms. The needle 16 can be coated with a substance 60 which can be released into the body upon insertion of the needle tip 18, as shown in FIG. 5.

The substance of various aspects of the invention is one that causes the skin to “plump” or swell. Non-limiting examples of substances include fats, silicone and stimulants. A specific aspect of the invention uses silicone as the substance. The substance is released from the needle upon one or more of physical manipulation of the needle, mechanical manipulation of the needle, by interaction of the substance with the body and by application of heat to either the needle or the region of the body.

Additional embodiments of the invention are directed to methods of administering acupuncture therapy where a needle is inserted into the skin of a patient and a substance is deposited under the skin. The substance deposited may have the effect of plumping the skin, or causing localized swelling.

The substance can be deposited by one or more of physical manipulation of the needle, mechanical manipulation of the needle, interaction of the substance with the body and application of heat to the target area of the body. In specific aspects, the substance is silicone. In other specific aspects, the substance is coated on the outside of a needle or within a syringe where the tip acts to puncture the skin.

In some aspects, the needle is inserted into the skin to a depth less than that which would contact a blood vessel. In detailed aspects, the insertion depth is determined by a sensor system capable of evaluating the depth at which blood vessels are found.

In further detailed aspects, the substance is automatically deposited by a pump mechanism coupled to the needle. The pump mechanism can be activated by the sensor system detecting that the needle has been inserted to the desired depth. Reference throughout this specification to “one embodiment,” “certain embodiments,” “one or more embodiments,” “an embodiment,” “one aspect,” “certain aspects,” “one or more embodiments” and “an aspect” means that a particular feature, structure, material, or characteristic described in connection with the embodiment is included in at least one embodiment of the invention. Thus, the appearances of the phrases such as “in one or more embodiments,” “in certain embodiments,” “in one embodiment,” “in an embodiment,” “according to one or more aspects,” “in an aspect,” etc., in various places throughout this specification are not necessarily referring to the same embodiment or aspect of the invention. Furthermore, the particular features, structures, materials, or characteristics may be combined in any suitable manner in one or more embodiments or aspects. The order of description of the above method should not be considered limiting, and methods may use the described operations out of order or with omissions or additions.

It is to be understood that the above description is intended to be illustrative, and not restrictive. Many other embodiments will be apparent to those of ordinary skill in the art upon reviewing the above description. The scope of the invention should, therefore, be determined with reference to the appended claims, along with the full scope of equivalents to which such claims are entitled.

As used in this specification and the appended claims, the singular forms “a”, “an” and “the” include plural referents unless the context clearly indicates otherwise. For example, reference to a “substance” may also refer to more than one substance, and the like.

What is claimed is:

1. An intelligent needle for subcutaneous injection, comprising:
a housing;
a movable needle within the housing, the needle having a tip and being movable between a retracted position where the needle tip is within the housing to an extended position where the needle extends from within the housing;
an adjustment mechanism coupled to the needle, the adjustment mechanism operative to move the needle between the retracted position and the extended position; and
a sensor operatively coupled to the adjustment mechanism, the sensor adapted to determine a penetration depth, the penetration depth being an amount the needle can penetrate the skin while avoiding blood vessels.

2. The needle of claim 1, further comprising a pump mechanism coupled to the needle and sensor, the pump mechanism operable to pump contents of the needle into the skin upon receiving a signal from the sensor.

3. The needle of claim 1, further comprising a substance which causes plumping of the skin.

4. The needle of claim 3, wherein the substance coats the needle.

5. The needle of claim 3, wherein the substance is prefilled in the needle.

6. The needle of claim 3, wherein the substance is silicone.

7. The needle of claim 3, wherein the substance is released from the needle upon one or more of physical manipulation of the needle, mechanical manipulation of the needle, interaction with the body and applied heat.

8. A method of administering acupuncture therapy, comprising:
inserting a needle into the skin of a patient; and
depositing a substance under the skin of the patient, the substance operative to cause plumping of the skin.

9. The method of claim 5, wherein the needle is inserted into the skin to a depth less than that which would be required to contact a blood vessel.

10. The method of claim 6, wherein the insertion depth is determined by a sensor.

11. The method of claim 5, wherein the substance is deposited by one or more of physical manipulation of the needle, mechanical manipulation of the needle, interaction with the body and application of heat.

12. The method of claim 5, wherein the substance is silicone.

13. The method of claim 5, wherein the substance is coated on the needle.

14. The method of claim 7, wherein the substance is automatically deposited by a pump coupled to the needle, the pump activated by the sensor detecting the needle inserted to the desired depth.
